(19) United States
(12) Patent Application Publication
(10) Pub. No.: US 2024/0024618 A1
(43) Pub. Date: Jan. 25, 2024

(54) BREATHWORK SYSTEMS AND METHODS

(71) Applicant: Yaymin, Inc., Frisco, TX (US)

(72) Inventors: Jaymin Shah, Frisco, TX (US); Yash Ghanekar, Frisco, TX (US)

(21) Appl. No.: 18/355,667

(22) Filed: Jul. 20, 2023

Related U.S. Application Data
(60) Provisional application No. 63/391,121, filed on Jul. 21, 2022.

(51) Int. Cl. A61M 21/02

(52) U.S. Cl. CPC A61M 21/02; A61M 2021/0022

(57) ABSTRACT

Breathwork systems and methods are provided which include a breathwork kit and a breathwork tool that guides a user through one or more breathwork sequences.

### RELATED APPLICATIONS

This application is based on, and claims benefit of and priority to, U.S. Provisional Patent Application Ser. No. 63/391,121 filed on Jul. 21, 2022, the contents of which are hereby incorporated by reference in their entirety for all purposes.

### BACKGROUND

Breathing exercises have been known in the art for thousands of years. Some forms of breathing exercises are instinctual for some. Perhaps some consciously or unconsciously change breathing and utilize this as a tool for depression, anxiety, exhaustion, fear, sadness, happiness, excitement, and/or preparation. In this way, conscious breathing has been a tool utilized by many for millenia.

Conscious breathing can be practiced in a variety of circumstances. Conscious breathing can be practiced for a variety of reasons. Conscious breathing helps a user control how much oxygen is reaching the lungs and thus can control how much oxygen is hitting cells and organs for improved function, presentness, and the like. Whether conscious breathing is utilized to help a user be present, think more clearly, even digest food or the like, conscious breathing is a proven tool for improvement in many circumstances. For example, one important compound in breathing and conscious breathing is carbon dioxide (CO2). CO2 has various effects on the body and control of CO2 and the release of carbon dioxide are well known breathing instruments and techniques in breathing and breathing exercises. By consciously controlling the exhale, a user can control the release of CO2 levels in the body and trigger the parasympathetic nervous system. One effect is that by releasing CO2, oxygen is actually caused to be taken into the bloodstream. So, this control of CO2 release and amounts of CO2 release can have many implications and impacts on the human body. For this reason, the control of the exhale can be a very important factor in breathing exercises. More specifically, overbreathing causes the body to lose considerable CO2. The loss of excess CO2 can cause side effects such as gasping, trembling, choking and a feeling of being smothered.

Practicing breathing, or breathing exercises, also known as conscious breathing is oftentimes controlling one's breath. This may be controlled by regulating how deep or shallow breaths are. This may also be controlling how quickly one is breathing, such as rate and the like. For example, a user may take long, deep breaths, and only through the nostrils. This generally causes the abdomen to expand before a breath fills the upper chest. This type of breathing can relax a user when patterned. This is one example of conscious breathing. There are many different purposes and types of conscious breathing.

Some experienced breathers who have been practicing breathing techniques for decades, or years, such as breathing instructors, are even able to identify tenses, ailments such as headaches, posture caused from breathing, emotions generated from breathing techniques, and the like. However, the inexperienced, or most of us are not able to easily identify personal body and/or mental states based solely on current breathing techniques.

Identifying breathing can be critical to physical and emotional states. Then, modifying this breathing can vastly improve the emotional and/or physical state of a user. However, most are not experienced in these identifications.

Thus, there is a long-felt need in the art for a convenient, easy to use apparatus and system which can help a user identify current breathing states, emotional states, physical states and the like. Furthermore, there is a long felt need in the art for a system which can then show a user how to regulate their breathing to improve upon such a state of emotional and/or physical stress, anxiety, or the like. Short of taking a breathing coach with a user everywhere they go, there is currently nothing available in the state of the art for a user to enhance their life, both physically and emotionally, at any time through breathing exercise and/or breathing regulation.

### SUMMARY OF THE INVENTION

Pursuant to some embodiments, breathwork systems, methods, devices and computer program code are provided. Pursuant to some embodiments, a breathwork tool includes a mouth, having a breath intake hole, a housing extending from a first end detachably connected to the mouth and a second end connected to an electrical connector, the housing having an interior space containing a rechargeable battery powering a memory storing executable program code and a processing unit to execute the program code to cause the breathwork tool to generate a first signal indicating a start of a breathwork sequence, generate a second signal indicating a first user action, generate a third signal indicating at least a second user action, and generate at least a fourth signal indicating a termination of the breathwork sequence.

In some embodiments, the breathwork tool further contains one or more sensors. In some embodiments, an air pressure sensor may be provided. In some embodiments, the breathwork tool may include a vibration motor and a light source to signal breathwork sequence information and other data to a user. In some embodiments, a signal may include the termination of a vibration, a vibration, a pattern of vibration, a light color, a light sequence, or a combination thereof.

Pursuant to some embodiments, a breathwork kit is provided which includes a breathwork tool and a case. In some embodiments the case may include a power source for charging a battery of the breathwork tool.

In some embodiments, a breathwork kit may include an application associated with a user device in communication with the breathwork tool via a wireless interface. Other features will become apparent upon reading the following disclosure.

### BRIEF DESCRIPTION OF THE DRAWINGS

FIGS. 1A-1F are views of a breathwork kit and case pursuant to some embodiments of the present invention;

FIG. 2 is an exploded view of a case pursuant to some embodiments;

FIG. 3 is a view of a breathwork tool pursuant to some embodiments;

FIG. 4 is a top view of a mouth of the breathwork tool of FIG. 3 pursuant to some embodiments;

FIG. 5 is an exploded view of the breathwork tool of FIG. 3 pursuant to some embodiments;

FIG. 6 is a block diagram depicting a breathwork system pursuant to some embodiments; and

FIG. 7 is a flow diagram depicting a breathwork process pursuant to some embodiments.

FIGS. 8A and 8B are illustrative user interfaces pursuant to some embodiments.

FIG. 9 is a partial cross sectional view of the breathwork tool of FIG. 3 pursuant to some embodiments.

### DETAILED DESCRIPTION

The following description is provided to enable any person in the art to make and use the described embodiments. Various modifications, however, will be readily-apparent to those in the art.

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure(s). The following detailed description is, therefore, not to be taken in a limiting sense, and the scope of the disclosure(s) is defined only by the appended claims, along with the full scope of equivalents to which such claims are entitled.

As used herein, the terminology such as vertical, horizontal, top, bottom, front, back, end, sides and the like are referenced according to the views, pieces and figures presented. It should be understood, however, that the terms are used only for purposes of description and are not intended to be used as limitations. Accordingly, orientation of an object or a combination of objects may change without departing from the scope of the disclosure.

Reference throughout this specification to “one embodiment,” “an embodiment,” “one example,” or “an example” means that a particular feature, structure, or characteristic described in connection with the embodiment or example is included in at least one embodiment of the present disclosure. Thus, the appearance of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Embodiments in accordance with the present disclosure may be embodied as an apparatus, method, or computer program product. Accordingly, the present disclosure may take the form of an entirely hardware-comprised embodiment, an entirely software-comprised embodiment (including firmware, resident software, micro-code, etc.), or an embodiment combining software and hardware aspects that may all generally be referred to herein as a “circuit,” “module,” or “system.” Furthermore, embodiments of the present disclosure may take the form of a computer program product embodied in any tangible medium.

Any combination of one or more computer-usable or computer-readable media may be utilized. For example, a computer-readable medium may include one or more of a portable computer removable drive, a hard disk, a random access memory (RAM) device, a read-only memory (ROM) device, an erasable programmable read-only memory (EPROM or Flash memory) device, a portable compact disc read-only memory (CDROM), an optical storage device, and a magnetic storage device. Computer program code for carrying out operations of the present disclosure may be written in any combination of one or more programming languages. Such code may be compiled from source code to computer-readable assembly language or machine code, or virtual code, or framework code suitable for the disclosure herein, or machine code suitable for the device or computer on which the code will be executed.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, and measured service), service models (e.g., Software as a Service (“Saas”), Platform as a Service (“PaaS”), and Infrastructure as a Service (“IaaS”)), and deployment models (e.g., private cloud, community cloud, public cloud, and hybrid cloud).

The flowchart and block diagrams in the attached figures illustrate the architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

In the arrangement shown, as one example, a stress and anxiety reduction system, a deep breathing tool, a haptic feedback system, and methods of use are presented. In some embodiments, a breathwork tool is provided which may be operated by a user to perform a breathwork sequence. In some embodiments, the breathwork tool prompts or signals the user to perform certain actions during a breathwork sequence (e.g., by vibrating or changing a light). The breathwork tool is easy to operate and easily held and carried, allowing a user to easily perform breathwork sequences to improve the user's mood, reduce anxiety, and obtain other beneficial results. In some embodiments, a breathwork tool includes a microprocessor and memory for storing and executing one or more breathwork sequences. Further, one or more sensors may be provided which monitor or otherwise detect the performance of a sequence (e.g., to detect a user's exhale, as well as the quality and duration of the exhale). In some embodiments, the breathwork tool may be placed in communication with a user device for a number of operations which will be described further below. Features of some embodiments will now be described by reference to the figures.

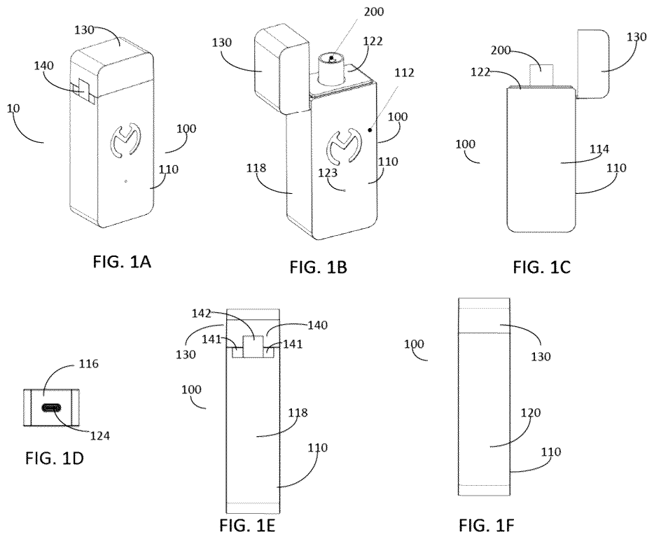

FIG. 1 depicts various views of a breathwork kit 10 which includes a case 100 and a breathwork tool 200 pursuant to some embodiments. Referring first to FIG. 1A, a breathwork kit 10 pursuant to some embodiments includes a case 100 that has a base 110 (which may also be referred to herein as a “charging case”) and a lid 130. The lid 130 is movably attached to the base 110 via a hinge 140 that allows the lid 130 to be positioned between a closed position (as shown in FIG. 1A) and an open position (as shown in FIG. 1B) which allows a user access to remove a breathwork tool 200 from the base 110. In some embodiments, other coupling devices may be used to allow the lid 130 to be positioned in an open position allowing a user to access the breathwork tool 200 (e.g., the hinge may be positioned on a different side of the base 110, or the lid 130 may be completely detachable from the base 110).

Pursuant to some embodiments, the hinge 140 is formed such that when the lid 130 is positioned in the opened position (as shown in FIG. 1B), the lid 130 is offset so that the bottom of the lid 130 extends substantially parallel to the side of the base 110. This allows easy removal of the breathwork tool 200 from the base 110. The hinge 140 has a central portion 142 that is longer than two side portions 141 as shown in FIG. 1E, allowing the lid 130 to be positioned in the open position shown in FIG. 1B.

In some embodiments, the case 100 is generally rectangular in shape, having a front face 112 (shown in FIG. 1B), a first side face 118 (shown in FIGS. 1B and 1E), a second side face 120 (FIG. 1F), a rear face 114 (FIG. 1C) and a bottom face 116 (FIG. 1D). In some embodiments, one or more of the faces 112-116 may be engraved or embossed with a logo or other ornamental markings. As shown in FIGS. 1A and 1B, the markings may be placed on a front face 112 of the case 100 (although other positions may also be used). The size of the case 100 is generally selected to allow the breathwork kit to be fit in a user's pocket (e.g., in some embodiments, the case 100 is approximately mm wide and 76.6 mm tall with a depth of 20 mm, although other shapes and dimensions may be used with desirable results). In some embodiments, the case 100 is formed of polycarbonate and ABS (a blend of a polymer such as acrylonitrile), although other materials may be used with desirable results so long as the case 100 is able to withstand repeated use and perform the functions described herein.

In some embodiments, the front face 112 may include a charging indicator 123 which allows a light emitting element within the base 110 to show a current charge status of a battery in the base 110. For example, the light emitting element may produce a first color of light when the battery in the base 110 is fully charged, and a second color of light when the battery in the base 110 is not fully charged. The battery in the base 110 (not shown in FIG. 1) allows the breathwork tool 200 to be charged as will be described further below. As shown in FIGS. 1A and 1B, the charging indicator 123 may be positioned on a front face 112 of the base 110, although other locations may also be used. The base 110 may also include a connector 124 (e.g., such as a USB connector and, in some embodiments as shown in the figures, a USB-C connector) that allows the base 110 to be connected to a power source (not shown) to charge a battery located within the base 110.

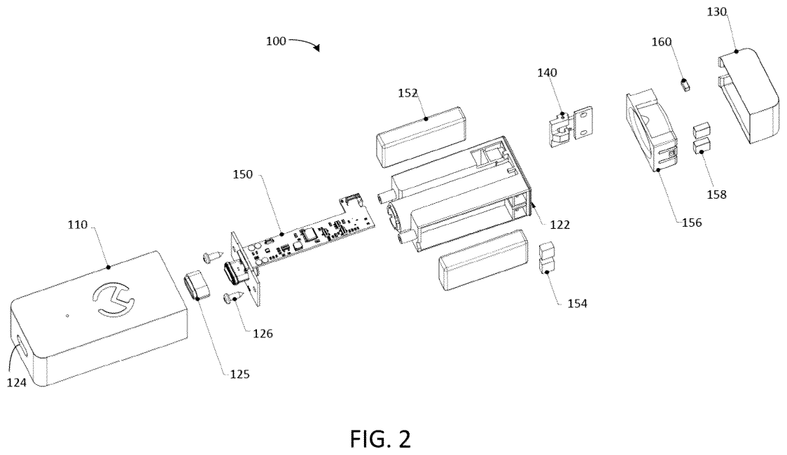

Pursuant to some embodiments, a top surface of the base 110 is formed with a base bracket 122 (shown in FIGS. 1B and 1C). The base bracket 122 is formed with an aperture to receive the breathwork tool 200, allowing the breathwork tool 200 to be slid into the base 110 for charging and storage, and to be slid out of the base 110 for use by a user. Pursuant to some embodiments, the base bracket 122 is magnetized such that when the lid 130 is positioned in the closed position (as shown in FIG. 1A), the magnetism of the base bracket 122 holds the lid 130 in the closed position. Further details of the base bracket will be described below in conjunction with a description of FIG. 2.

Pursuant to some embodiments, the case 100 includes a lid 130. Lid 130 is formed of any suitable size, shape, and design and is configured to provide access to the components within and particularly a breathwork tool 200 (to be further described herein). In the arrangement shown in FIG. 1, as one example, the lid 130 includes a hollow interior and is hingedly connected such that the lid 130 can pop open at the press of a magnetic, frictional or physical engagement. In some embodiments, as shown, the lid 130 is shaped to be familiar to daily users as a common household item (e.g., such as a lighter) and is designed to withstand continual use, even by a fidgety user. Pursuant to some embodiments, the lid 130 is magnetically engaged with the base 110. Lid 130 may also be attached through a screw-like feature, a clip, or other connections as a means of attaching and/or removing the lid 130 from the case 100. Lid 130 is generally located at the top or first end of the base 110 of the case 100.

Reference is now made to FIG. 2 which is a partial exploded view of the case 100. The case 100 includes a base 110 and a number of components that fit inside the base 110, as well as a lid 130 and a number of components that fit inside the lid 130. For example, in some embodiments, the base 110 includes a connector mouth 125 (that fits inside the connector 124 allowing a power source to be inserted into the connector mouth 125). The connector mouth 125 is in electrical communication with a charging case circuit board 150 that distributes power to circuitry for use in charging one or more charging case batteries 152 and for providing charging power to a battery of the breathwork tool 200 (not shown in FIG. 2). In some embodiments, the batteries 152 may be 310 mAh lithium ion polymer batteries, although other types and sizes of batteries may be used so long as the batteries are capable of charging the breathwork tool 200 for use as described herein. The charging case circuit board 150 may also include circuitry to provide power to a charge indicator 123 (FIG. 1B) light source on the charging case circuit board 150 that emits light to indicate a status of a charge of the charging case batteries 152. The charging case circuit board 150 may be held in place within the base 110 via a connection between the connector mouth 125 and the connector 124 of the base 110.

The connector mouth 125 and connector 124 (and the power connection in general) may be formed of any suitable size, shape, and design. In the arrangement shown, as one example, the power connection is formed of a connector mount 125 and connector 124 are positioned for use with a power cord (not shown) which can be connected and which can subsequently be plugged into an power point or outlet. However, an access panel to one or more batteries 152 or other power supply may also be used.

The charging case circuit board 150 may be connected to a base bracket 122 via one or more screws 126. The base bracket 122 may be formed with a central recess that is shaped to receive the breathwork tool 200 (not shown in FIG. 2). When the breathwork tool 200 is inserted through the base bracket 122 it may be placed in electrical connection to the charging case circuit board 150 so that a battery of the breathwork tool 200 (not shown in FIG. 2) may be charged. The end of the base bracket 122 is formed of a material that allows magnetic fields from one or more magnets 154 to be formed on a top surface of the base bracket 122 (the surface that faces the lid 130). The inventors have found that by placing several magnets 154 at an outer edge of the top surface of the base bracket 122, that desirable results may be achieved. For example, by placing the magnets 154 near a side of the base 110 away from a hinge 140, that a better connection between the base 110 and the lid 130 may be obtained. Further, by placing the magnets 154 near the side of the base 110, the magnetic field does not disrupt the breathwork tool 200 as it is inserted into the base bracket 122.

The lid 130 encloses several items. For example, in some embodiments, the lid 130 encloses a lid bracket 156 which is formed to fit within a bottom of the lid 130 and which holds a hinge magnet 160 which is positioned proximate the hinge 140 that attaches the lid 130 to the base 110. The lid bracket 156 also holds one or more lid magnets 158 which are positioned to be proximate the base magnets 154 when the lid 130 is in a closed position (e.g. as shown in FIG. 1A).

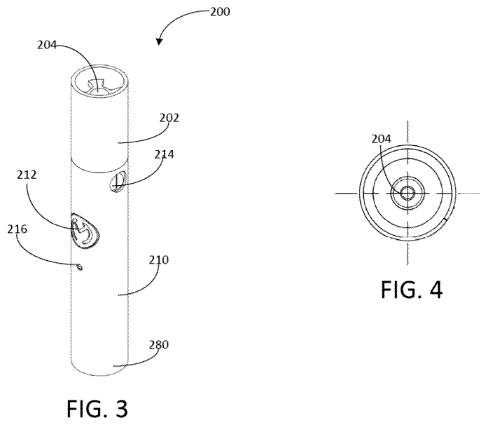

Reference is now made to FIG. 3 where a perspective view of a breathwork tool 200 pursuant to some embodiments is shown. As described above, the breathwork tool 200 is shaped and sized to fit within the base 100 for charging and storage.

In the arrangement shown, as one example, the breathwork tool 200 includes a housing 210. Housing 210 is formed of any suitable size, shape, and design and is configured as the primary body and breathing apparatus of the present disclosure. The breathwork tool 200 extends a length from a first end (having a connector 280) to a second end (having a mouth 202). Pursuant to some embodiments, the breathwork tool 200, mouth 202 and housing 210 are primarily cylindrical in shape with a tube-like appearance. However, other shapes and sizes are also hereby contemplated for use. In use, a user exhales into an intake 204 of the mouth 202 during a breathwork sequence guided by the breathwork tool 200 as described further herein. The user's actions (such as inhaling, exhaling into the intake 204, and performing breath holds) are guided by indicators provided by the breathwork tool 200 (e.g., such as a haptic vibration, a light signal emitted from an light or light emitting diode “LED” aperture 216, etc). The use of these indicators will be described further herein.

In the arrangement shown in FIG. 3, the mouth 202 is removably attached to the housing 210 (e.g., for cleaning). For example, the mouth 202 and the top of the housing 210 may be threaded, allowing the mouth 202 to be unscrewed from the housing 210. Further, pursuant to some embodiments, the intake 204 of the mouth 202 is formed in such a way to maximize the air flow into the housing 210 (during a user's exhale) while minimizing the amount of moisture that enters the housing 210.

The housing 210 includes a button 212, one or more air holes 214, and an LED aperture 216. The button 212 may be depressed by a user to initiate operation of the breathwork tool 200 as will be described further herein. The one or more air holes 214 allow air to escape from the breathwork tool 200 when a user of the breathwork 200 exhales during a breathwork exercise as will be described further herein. Housed within the housing 210 are a various components, as are further described herein, for the operation of the breathwork tool 200.

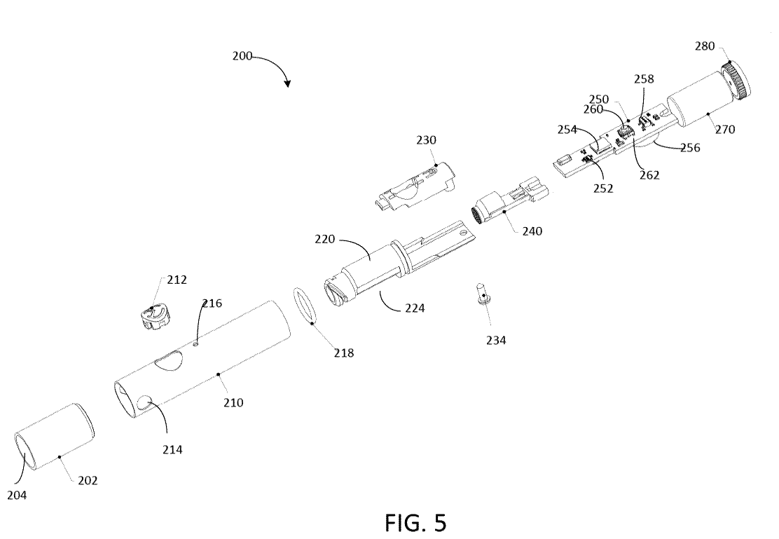

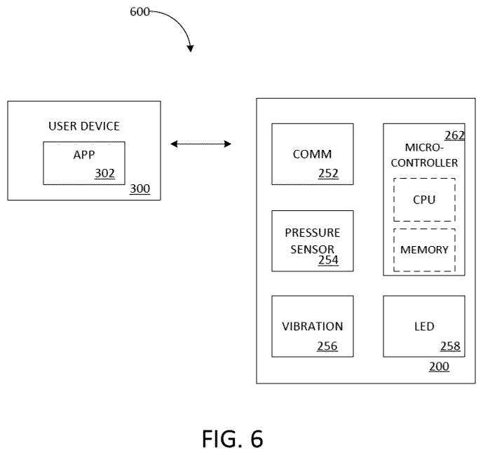

Referring to FIG. 4, a top view of the breathwork tool 200 is shown which shows an intake 204 formed in the center of the mouth 202. The intake 204 allows a user to exhale into the mouth 202 of the breathwork tool 200 during breathwork exercises. In some embodiments, the intake 204 is formed with a center section that allows air to enter the interior of the breathwork tool 200 for routing toward the air pressure sensor (e.g., item 254 as shown in FIG. 5) and a series of air holes that allow air to enter the interior of the breathwork tool 200 for routing toward an air hole 204 (as shown in FIGS. 3 and 5). In this manner, users may comfortably exhale into the breathwork tool 200 without pressure in the tool building to a point where the exhale is impeded (as most of the air passes out of the breathwork tool 200 via the air hole 204).

Reference is now made to FIG. 5 where a partial exploded view of a breathwork tool 200 pursuant to some embodiments is shown. The breathwork tool 200 includes a number of components, including a mouth 202 (with an intake 204). The mouth 202 is removably attached to one end of a housing 210. The housing 210 includes one or more air holes 214, a button 212 and a led aperture 216. In some embodiments, an o-ring 218 is provided that fits within an interior of the housing 210 and provides an air and moisture seal between the mouth 202 and components that slide into the interior of the housing 210. The components that slide within the interior of the housing 210 include a first bracket 220, a second bracket 230, a silicon plug 240, a printed circuit board assembly 250 and a rechargeable battery 270. The silicon plug 240 prevents moisture from reaching the components mounted on the printed circuit board assembly 250. The first bracket 220, second bracket 230, silicon plug 240 and printed circuit board assembly 250 are connected using a screw 234 when the components are aligned for insertion into the housing 210. The bottom of the housing 210 (and the components therein) is sealed by a connector 280.

The rechargeable battery 270 is charged, in some embodiments, when the breathwork tool 200 is placed inside the base 110. For example, the rechargeable battery 270 may be charged by induction charging, by contact charging or the like. The connector 280 provides electrical contact between the battery 270 and the power supplied by the base 110 (e.g., via the batteries 152 or other power source). In some embodiments, the rechargeable battery 270 may be a 60 mAh lithium ion polymer battery, although other types and sizes of batteries may be used so long as the battery is capable of powering the breathwork tool 200 for use as described herein.

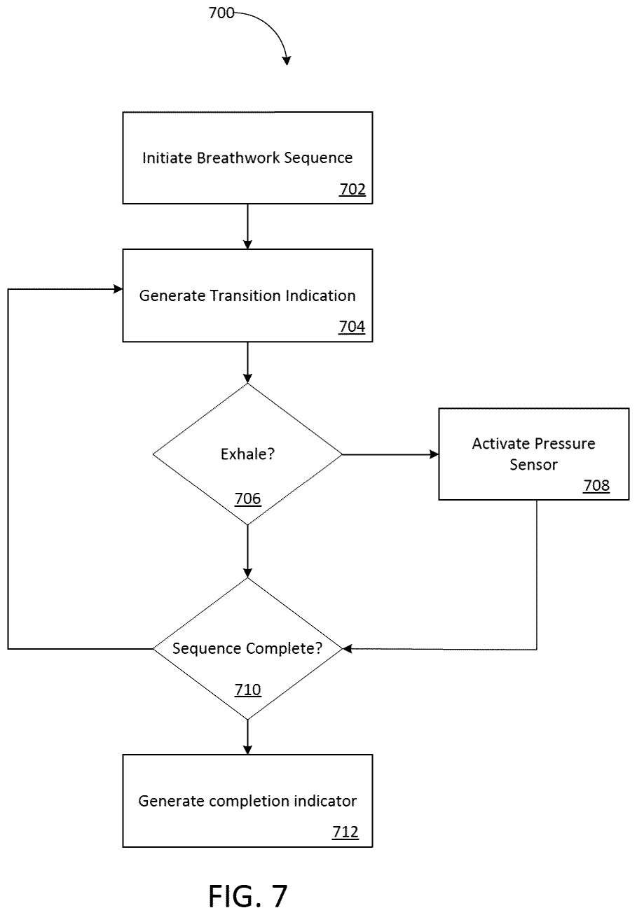

The printed circuit board assembly 250 includes a number of components, including, in some embodiments, a Bluetooth module 252 (for sending and receiving data from a user device such as a mobile phone as shown in FIG. 6), an air pressure sensor 254 for sensing the air pressure within the breathwork tool 200 when a user exhales into the tool), a vibration motor 256 for generating haptic signals, a light source 258 (for providing a visual indication through the aperture 216 of the operating state and/or battery charge of the breathwork tool 200), a power switch 260 (which is activated by a user pressing a button 212) and a microcontroller 262 (having, for example, an integrated processor and memory) for storing breathwork sequence information to control the operation of the breathwork tool 200 as will be described further below. Pursuant to some embodiments, the microcontroller 262 is an 8-bit microcontroller on a single chip (e.g., based on a 8051 processor architecture), with flash program memory and read only memory. In some embodiments, the microcontroller 262 is a low power consumption microcontroller having a small form factor.

The air hole 214 allows a user's exhale to send air into the housing 210 and is directed toward the printed circuit board 250 (and a pressure sensor 254 mounted thereon). Pursuant to some embodiments, the air may escape the interior of the housing through the air holes 214. Pursuant to some embodiments, the air is directed from the air hole 214 toward the pressure sensor 254 via an air escape path 224 formed in the first bracket 220 (the air escape path 224 is formed in the first bracket 220 to direct air towards the air pressure sensor 254 to ensure a reliable pressure reading). For example, in some embodiments such as shown in FIG. 5, the first bracket 220 is formed such that it has a cylindrical cross section at the point where it mates with the mouth 202, and then has an opening in the bottom of the cylinder to form an air escape path 224. This allows air to pass into the first bracket 220 even when the other components (such as the printed circuit board assembly 250) are inserted into the interior of the first bracket 220. Further, the pressure sensor 254 is mounted on the printed circuit board assembly 250 such that the pressure sensor 254 is directly exposed to the air passing into the first bracket 220. This ensures that reliable air pressure readings are taken when the breathwork tool 200 is used. Because the interior of the housing 210 is filled with a number of components (including the printed circuit board assembly 250 and electronics mounted thereon) it is difficult to ensure that sufficient air flow is provided during a user's exhale into the breathwork tool 200. Some embodiments include an air escape path 224 configured to ensure that the air flow during a user's exhale is directed toward the air pressure sensor 254 to enable the air pressure sensor 254 to reliably information associated with the exhale. The air pressure sensor 254 may generally be used to measure data about a user's exhale including, for example, data associated with air flow amount, speed, direction, and the like.

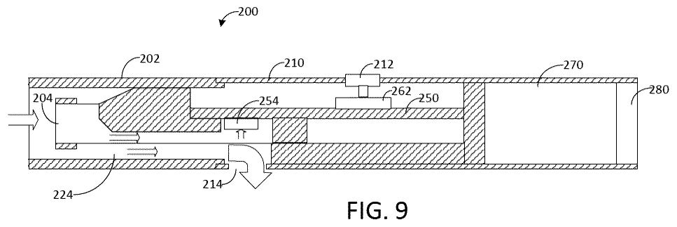

A cross sectional view of the breathwork tool 200 pursuant to some embodiments is shown in FIG. 9. The view of FIG. 9 is not to scale and does not show all of the internal components of the breathwork tool 200 and is intended to primarily depict the paths that an exhale into the tool take. As shown in FIG. 9, the breathwork tool 200 includes a mouth 202 and a housing 210. The mouth 202 includes an intake 204. When a user exhales into the breathwork tool 200 (e.g, by placing their lips around the mouth 202), air passes into the breathwork tool 200 along two paths. A first path is entered via the center of the intake 204 and allows some air to pass into an interior of the breathwork tool 200 towards an air pressure sensor 254. This allows the air pressure sensor 254 to detect the exhale and measure attributes of the exhale (including, for example, the duration and consistency of the exhale). A second path is entered via a series of air holes or other apertures along an outer circumference of the intake 204. This air path directs the exhale out of the breathwork tool 200 via an air escape path 224 and out one or more air holes 214. The combination of these paths allow a user to perform a exhale without being impeded by built up air pressure within the breathwork tool 200.

Referring again to FIG. 5, pursuant to some embodiments, the breathwork tool 200 provides a timing operation (generally referred to herein as a “breathwork sequence”). The breathwork sequence is controlled by signals emitted by the microcontroller 262 pursuant to a specific selected sequence. For example, the microcontroller 262 may issue one or more signals to operate the vibration motor 256 to cause a haptic vibration to occur which can be felt by the user holding the breathwork tool 200. The microcontroller 262 may issue a different signal to operate the LED 258 (e.g., to cause the LED to emit a different color, or to blink, etc.). These signals may be issued on a timed sequence pursuant to a selected breathwork sequence as described further below. In this way, the system can provide timed breathing and the like and help a user regulate their breathing based on timing sequences and more. A user can be guided through the breathwork sequence by a vibration generated by the breathwork tool 200 (e.g., a “haptic” vibration). In one example, breathwork tool 200 provides a single haptic vibration per second of inhalation and a long continuous vibration for exhaling. This is one example of haptic feedback. However, various timing and vibration lengths are used for various exercises and the like.

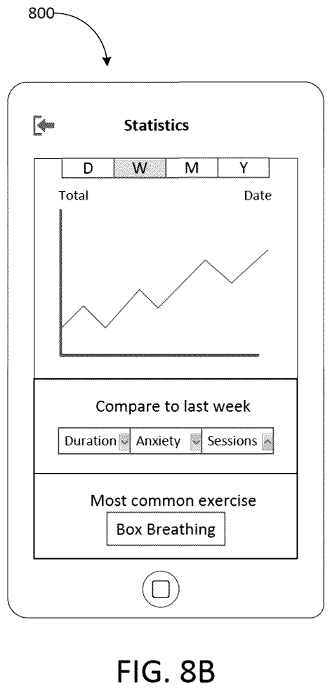

In one mode of operation, the breathwork tool 200 is configured to operate to guide a user to perform a so-called “perfect exhale” (which is an even exhale of 5.5 seconds in duration). In this mode of operation, the breathwork tool 200 directs a user through a specific breathwork sequence (e.g., which may be stored in a memory 262 of the breathwork tool 200) to train the user. The sequence, pursuant to some embodiments, operates as follows. The user initiates the sequence by removing the breathwork tool 200 from the case 100 and exhaling into the intake 204 on the mouth 202 of the breathwork tool 200. The air pressure sensor 254 in the housing 210 detects that an exhale is being performed and initiates a timing sequence. The air pressure sensor 254 continues to measure the air pressure during the timing sequence. After 5.5 seconds has elapsed, a signal (such as a haptic vibration caused by the vibration motor 256) indicates the end of the breathwork sequence to the user. In some embodiments, the LED 258 may also provide some feedback to the user (e.g., by displaying a color that signals to the user that the exhale is being performed properly, or a different color to signal that the exhale is not being performed properly). Those skilled in the art, upon reading this disclosure, will appreciate that a number of other breathwork sequences and feedback signals may be used to guide a user through a breathwork session. Once the sequence is initiated, the user performs an exhale into the breathwork tool 200. The air pressure sensor 254 detects and measures the exhale and tracks the air pressure (to ensure the exhale is even in pressure throughout the sequence) and tracks the duration of the exhale. A visual indicator (such as a blue light) is displayed through the aperture 216 during the exhale and the vibration motor 256 vibrates twice (providing a double haptic feedback signal to the user) at the end of a 5.5 second exhale. By repeating this sequence, a user can be trained to improve their exhale to achieve a “perfect exhale”. In some embodiments, when used in conjunction with a user device (such as a mobile application as discussed below in conjunction with FIG. 6), the user may track their progress in achieving a perfect exhale. For example, in some embodiments, graphical depictions of user interfaces pursuant to some embodiments showing a user's progress are shown in FIGS. 8A and 8B.

Examples of other breathwork sequences that may be guided using the present invention are shown below. Those skilled in the art, upon reading the present disclosure, will appreciate that a number of other sequences may also be provided (and users operating the breathwork tool 200 in conjunction with a paired user device such as shown in FIG. 6 may configure custom breathwork sequences as well). Table 1 below shows a guided sequence for a pre-set sequence that may be stored in a memory 262 of the breathwork tool 200 which is referred to as a “box breathing” sequence.

TABLE 1

| User Action | Indicator | Duration |
|---|---|---|
| Inhale | One haptic vibration per second | 4 seconds |
| Breath Hold | No vibrations | 4 seconds |
| Exhale | Continuous vibration | 4 seconds |
| End of sequence | Vibrations stop | — |

Table 2 below shows another breathwork sequence that may be guided using the breathwork tool 200. This sequence is referred to as “4-4-8 breathing”.

TABLE 2

| User Action | Indicator | Duration |
|---|---|---|
| Inhale | One haptic vibration per second | 4 seconds |
| Breath Hold | No vibrations | 4 seconds |
| Exhale | Continuous vibration | 8 seconds |
| End of sequence | Vibrations stop | — |

Pursuant to some embodiments, a user may operate the breathwork tool 200 as follows. First, the user removes the breathwork tool 200 from the case 100 (after confirming that the breathwork tool 200 is sufficiently charged by reference to the light emitted via the led aperture 216). The user depresses the button 212 to initiate a breathwork sequence (which may be a sequence stored in a memory of the breathwork tool 200 and discussed in conjunction with FIG. 5 below, or a sequence received from a mobile device as discussed in conjunction with FIG. 6 below). The user will inhale, exhale and perform breath holds as directed by vibrations of the breathwork tool 200 (as discussed further below). When the user exhales, the user will exhale into the mouth 202 of the breathwork tool 200 which allows the breathwork tool 200 to detect the pressure at which the user is exhaling (as discussed further below). In some embodiments, a user may perform breathwork exercises without the breathwork tool 200 in communication with a mobile device (e.g., using breathwork sequences stored in a memory of the breathwork tool). In some embodiments, a user may perform breathwork exercises that are administered by the breathwork tool 200 based on sequences provided to the breathwork tool 200 from a mobile device.

Pursuant to some embodiments, the breathwork tool 200 is able to provide lung capacity metrics and feedback. For example, lung capacity metrics are measured through pressure sensors (shown in FIG. 5) and the like to provide a set of parameters for lung function, deep breathing and feedback and exercises to increase lung capacity. This may include but not limited to, increasing oxygen to muscles and other organs for better performance and strengthening of respiratory muscles.

The breathwork tool 200 may include other sensors (not shown). These and other sensors may be included therein as part of the onboard computing system (to be further described herein). In some embodiments, by example, a carbon dioxide or “CO2” sensor is included. The CO2 sensor detects and/or measures carbon dioxide levels. These levels are correlated to anxiety and the like. Utilizing the measurements and/or parameters of these levels along with at least one CO2 sensor provides for various functionality and the like, which is further described herein, including but not limited to, changes in coaching and the like.

Reference is now made to FIG. 6 where a breathwork system 600 pursuant to some embodiments is shown. The breathwork system 600 includes the breathwork tool 200 and a user device 300 which are in communication with each other (e.g., via a Bluetooth interface or the like). For example, the user device 300 may be a mobile device such as a smartphone (e.g., such as a mobile device operating the iOS or Android operating systems or the like). The user device 300 may have a breathwork application 302 installed thereon which allows the user device 300 to send and receive information to the breathwork tool 200. For example, in some embodiments, a user may interact with the breathwork application 302 to select a breathwork sequence to perform. Information identifying the breathwork sequence may be transmitted to the breathwork tool 200 (which receives the information via the communication port 252 which may be, for example, the Bluetooth chip 252 of FIG. 5).

The breathwork tool 200 may store the sequence data in a memory of microcontroller 262 (which may be, for example, the microcontroller 262 of FIG. 5). When the user is ready to perform the sequence, the user may activate the breathwork tool 200 by depressing or otherwise activating the button 212 on the tool. The microcontroller 262 executes program code to perform the sequence and issues control signals to control the LED 258 and/or the vibration motor 256 to provide visual and/or haptic vibration cues to a user to guide the user through the breathwork sequence as described above. Further, pressure information detected by the pressure sensor 254 may be captured by the microcontroller 262 and transmitted to the user device 300 for further analysis by the user device 300 (e.g., to calculate a user's lung capacity, to track progress, or the like). In this manner, the breathwork system 600 can allow a user device 300 to be used in conjunction with the breathwork tool 200 to track progress, analyze user data, generate breathwork sequences, etc. In some embodiments, the microcontroller 262 may analyze the pressure information detected by the pressure sensor 254 to provide visual or haptic feedback to the user regarding the status of a breathwork sequence. For example, in some sequences, such as the “perfect exhale” sequence described above, the microcontroller 262 may change a color of light emitted from the LED 258 to signal to the user that the user is exhaling properly.

In some embodiments, the breathwork tool 200 may be used without communication with a user device 300. For example, the memory of the microcontroller 262 may store one or more breathwork protocols that may be followed without communication with a user device 300.

In some embodiments, the system 600 may also include one or more remote servers, databases, and/or computers that fulfill the functions disclosed and described herein. For example, an application server may be provided that is adapted to transmit and receive data from different user devices 300 regarding selected datasets related to various users and/or datasets related to multiple users. While the use of a microcontroller 262 with memory and a processor on a single chip are described, other embodiments may use a separate processor and memory rather than a single chip microcontroller.

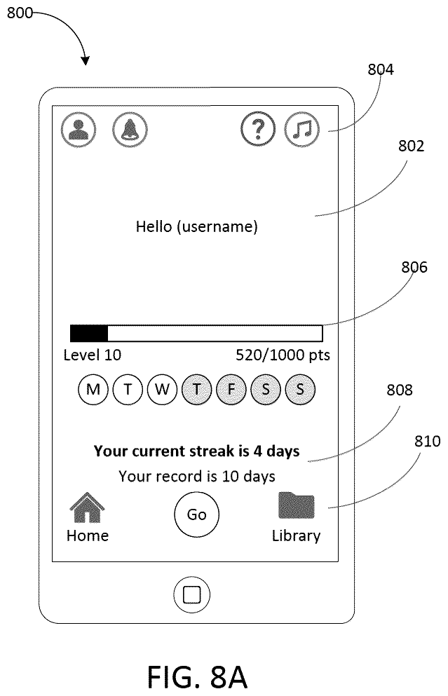

Reference is now made to FIG. 7 where breathwork process 700 pursuant to some embodiments is shown. The breathwork process 700 may be performed by a user operating the breathwork tool 200 (either alone or in communication with a user device 300). Process 700 begins at 702 where the user initiates a breathwork sequence. This may be done by the user depressing the button 212 on the breathwork tool 200 (and/or by initiating the sequence from a user device 300 in communication with the breathwork tool 200). Initiation at 702 may further include the user selecting a specific breathwork sequence from among a number of different sequences.

Processing continues at 704 where the user is prompted (e.g., by a haptic vibration) to perform a specific action (e.g., exhale, inhale, or breath hold). At 704, the microcontroller 262 issues a signal to cause the breathwork tool 200 to indicate to the user to perform a specific action (e.g., such as the signals and indications described in Tables 1 and 2 above). Depending on the specific sequence, each action may be performed for a predetermined amount of time (e.g., 4 seconds or the like). If the action is an exhale, processing continues at 708 where the pressure sensor 254 is activated to measure the air pressure within the breathwork tool 200. In some embodiments, the pressure sensor 254 may remain active throughout the sequence (e.g., for inhale sequences as well as for exhale sequences) and the pressure data is only saved for exhale sequences. If a determination is made that the sequence is not complete (at 710), processing continues at 704 where another transition indication is generated (e.g., such as a haptic signal indicating that the user is to perform the next action in the sequence). The process 700 continues until the breathwork sequence is completed. In some embodiments, upon completion of a sequence, a different signal (such as a haptic vibration) is generated informing the user that the sequence is complete. Processing at 712 may include operating the breathwork tool 200 to automatically save information from the sequence (e.g., such as the air pressure measurements, the number of iterations of the sequence completed, etc.). This information may be transmitted to a user device 300 for evaluation and reporting of progress to the user.

In this way, the present disclosure provides the state of the art with a haptic feedback exercise device capable of providing various exercises based on current state and needs of a user. The present disclosure provides a pre-set and machine learning set of exercises which provide guidance and rules indicating when to inhale, hold, and/or exhale. The present disclosure also provides for various breathing exercises and communication systems to indicate various commands. The present system is compact and easy to carry and use.

In some embodiments, a user device (such as the user device 300 of FIG. 6) may be paired or in communication with a breathwork tool 200. When in communication, the user device may be used by a user to interact with the breathwork tool 200 and/or to view data associated with the user's use of the breathwork tool 200. Example user interfaces of some embodiments are shown in FIGS. 8A and 8B. For example, as shown in FIG. 8A, a user interface 800 may be presented which allows a user to view statistics and information associated with their usage of the breathwork tool 200. As depicted, in some embodiments, data may be presented associated with a user's repeated usage of the breathwork tool 200 (e.g., to show information about a streak or pattern of use). In the illustrative embodiment shown in FIG. 8A, a user is presented with information showing the user's current streak of usage 806 and 808. This data may be used to gamify or incentivize the user's continued usage of the breathwork tool 200. The user may also be provided with navigational icons 804 allowing the user to navigate throughout the application (e.g., to view help resources, modify their user profile, receive notifications, etc.). The user may also be able to interact with icons 810 to view different breathwork protocols from a library of protocols, initiate a breathwork session (e.g., by clicking the “go” button) or navigate to a home screen.

As shown in the illustrative embodiment of FIG. 8B, a user interface 800 may allow a user to view data or other statistics about their usage and progress using the breathwork tool 200. In some embodiments, a user may view statistics and information associated with individual protocols (as shown, the user is viewing statistics about their usage of a box-breathing protocol). The user interfaces of FIGS. 8A and 8B are illustrative, and those skilled in the art, upon reading the present disclosure, will appreciate that a number of other user interfaces may be provided. For example, user interfaces may be provided to display information to users during the performance of a breathwork exercise. Such interfaces may include breathwork games that guide a user through proper breathing exercises with the air pressure sensor 254 verifying if the user is exhaling at the correct time(s) during a sequence. A user may be rewarded with feedback, points, or rewards (such as coupons or discounts) when an exercise is performed correctly. In some embodiments, the user device 300 may display a user interface during the performance of a breathwork sequence, providing a visual representation of when to inhale and exhale (thereby augmenting the haptic feedback signals provided by the breathwork tool 200). In some embodiments, the user device 300 may display an interface providing one or more wellness check forms, where the user is prompted to input information about the user's mood, anxiety level, caffeine intake, exercise status, etc. These data points may be used in conjunction with breathwork data to provide daily or other regular feedback to the user. In some embodiments, the user device 300 may include a library of breathwork exercises grouped in categories that may be selected by a user to achieve a specific outcome (such as calming exercises, exercises to increase energy levels, exercises to perform in the morning, exercises to perform to induce sleep, etc.). The application 302 may also issue reminders or notifications to the user to remind the user to perform breathwork exercises. In some embodiments, the user device 300 may display a visual representation of breath through an avatar. For example, in some embodiments, a user's breath is used as a controller in which the exhale controls animations within the application 302. As a more specific example, a user's exhale may cause an object in a game to rise (e.g., in a helicopter game, an exhale may lift the helicopter thereby teaching breathwork in a gamified way). The characters on a display screen of the user device 300 may also be interactive, and as a user exhales into the breathwork tool 200 elements are released from them to visualize the exhale in real time. In some embodiments, different elements of the application 302 may be directly interacted with via the user's breath. The application 302 may also provide the user with recommendations and guided playlists of different breathwork exercises.

The foregoing diagrams represent logical architectures for describing processes according to some embodiments, and actual implementations may include more or different components arranged in other manners. Other topologies may be used in conjunction with other embodiments. Moreover, each component or device described herein may be implemented by any number of devices in communication via any number of other public and/or private networks. Two or more of such computing devices may be located remote from one another and may communicate with one another via any known manner of network(s) and/or a dedicated connection. Each component or device may comprise any number of hardware and/or software elements suitable to provide the functions described herein as well as any other functions. For example, any computing device may include a programmable processor to execute program code such that the computing device operates as described herein.

All systems and processes discussed herein may be embodied in program code stored on one or more non-transitory computer-readable media. Such media may include, for example, a DVD-ROM, a Flash drive, magnetic tape, and solid state Random Access Memory (RAM) or Read Only Memory (ROM) storage units. Embodiments are therefore not limited to any specific combination of hardware and software.

Elements described herein as communicating with one another are directly or indirectly capable of communicating over any number of different systems for transferring data, including but not limited to shared memory communication, a local area network, a wide area network, a telephone network, a cellular network, a fiber-optic network, a satellite network, an infrared network, a radio frequency network, and any other type of network that may be used to transmit information between devices. Moreover, communication between systems may proceed over any one or more transmission protocols that are or become known, such as Asynchronous Transfer Mode (ATM), Internet Protocol (IP), Hypertext Transfer Protocol (HTTP) and Wireless Application Protocol (WAP).

Embodiments described herein are solely for the purpose of illustration. Those in the art will recognize other embodiments may be practiced with modifications and alterations to that described above.

What is claimed is:

1. A breathwork tool, comprising:
a mouth, having a breath intake hole;
a housing extending from a first end detachably connected to the mouth and a second end connected to an electrical connector, the housing having an interior space containing a rechargeable battery powering a memory storing executable program code and a processing unit to execute the program code to cause the breathwork tool to
generate a first signal indicating a start of a breathwork sequence;
generate a second signal indicating a first user action;
generate a third signal indicating at least a second user action; and
generate at least a fourth signal indicating a termination of the breathwork sequence.

2. The breathwork tool of claim 1, wherein the interior space further contains at least a first wireless communication port for transmitting and receiving data between the processing unit and an external user device.

3. The breathwork tool of claim 1, wherein the interior space further contains (i) a vibration motor and at least one of the signals controls operation of the vibration motor, and (ii) a light source and at least one of the signals controls operation of the light source.

4. The breathwork tool of claim 1, wherein the interior space further contains an air pressure sensor, the air pressure sensor operating with the processing unit to monitor the air pressure within the housing.

5. The breathwork tool of claim 4, wherein the interior space further contains a first air path and a second air path, the first air path directing air from the breath intake hole to an air hole formed in the housing and the second air path directing air from the breath intake hole to a surface of the air pressure sensor.

6. The breathwork tool of claim 1, further comprising:
a switch, electrically connected to the processing unit, the switch activating or deactivating operation of the breathwork tool.

7. The breathwork tool of claim 2, wherein the processing unit is further configured to execute program code to cause the breathwork tool to:
receive a breathwork sequence from the external user device; and
store the breathwork sequence in the memory.

8. The breathwork tool of claim 7, wherein the processing unit is further configured to execute program code to cause the breathwork tool to:
execute the stored breathwork sequence.

9. The breathwork tool of claim 2, wherein the processing unit is further configured to execute program code to cause the breathwork tool to:
capture air pressure data from an air pressure sensor; and
transmit the air pressure data to the external user device.

10. The breathwork tool of claim 1, wherein the second signal is a vibration each second for a period of time and the first user action is an inhale.

11. The breathwork tool of claim 1, wherein the third signal is a continuous haptic vibration for a second period of time and the second user action is an exhale.

12. The breathwork tool of claim 11, further comprising:
capturing air pressure data during the second period of time to measure information associated with the exhale.

13. The breathwork tool of claim 1, wherein the electrical connector connects the rechargeable battery to a case for charging.

14. A method for operating a breathwork tool to perform a breathwork sequence, the method comprising:
activating the breathwork tool;
selecting the breathwork sequence;
determining, based on the breathwork sequence, a number of iterations to be performed;
generating a first indicator instructing a user to perform a first user action pursuant to the selected breathwork sequence;
generating a second indicator instructing the user to perform a second user action pursuant to the selected breathwork sequence;
generating a third indicator instructing the user to perform a third user action pursuant to the selected breathwork sequence;
repeating the steps of generating until the number of iterations has been performed; and
storing the results of the user actions.

15. The method of claim 14, further comprising:
operating at least a first sensor to monitor the performance of at least one of the first user action, the second user action and the third user action.

16. The method of claim 15, wherein the at least first sensor is an air pressure sensor, and at least one of the first user action, the second user action and the third user action is an exhale.

17. A breathwork system, comprising:
a breathwork tool having a mouth with an air intake hole, a housing extending from a first end detachably connected to the mouth and a second end connected to an electrical connector, the housing having an interior space containing a rechargeable battery powering a microcontroller and an air pressure sensor, the interior space including a first airway for directing air from the air intake hole to an air hole formed in the housing, and a second airway for directing air from the air intake hole to a surface of the air pressure sensor; and
a case having a power source and a connector, the case having an interior for receiving the breathwork tool for charging a battery of the breathwork tool via the connector.

18. The breathwork system of claim 17, wherein the interior space of the breathwork tool further contains a Bluetooth communication device for transmitting breathwork data to a user device.
